(19) United States
(12) Patent Application Publication
(10) Pub. No.: US 2023/0177690 A1
(43) Pub. Date: Jun. 8, 2023

(54) IMAGE PROCESSING APPARATUS, IMAGE PROCESSING METHOD, AND STORAGE MEDIUM

(71) Applicant: CANON KABUSHIKI KAISHA, Tokyo (JP)

(72) Inventor: TAKUYA KUBO, Kanagawa (JP)

(21) Appl. No.: 18/155,277

(22) Filed: Jan. 17, 2023

Related U.S. Application Data
(63) Continuation of application No. PCT/JP2021/025782, filed on Jul. 8, 2021.

(30) Foreign Application Priority Data
Jul. 20, 2020 (JP) 2020-123747
Jun. 23, 2021 (JP) 2021-104154

(51) Int. Cl. G06T 7/00; H04N 5/265; G06V 10/764; G16H 15/00

(52) U.S. Cl. CPC G06T 7/0012; G06V 10/764; G16H 15/00; H04N 5/265; G06T 2207/10056; G06T 2207/20104; G06T 2207/30024; G06V 2201/07

(57) ABSTRACT

An image processing apparatus includes an acquisition unit configured to acquire image data as a result of imaging a Gram-stained specimen, and a generation unit configured to generate a display image by superimposing a position where a bacterium classified by Gram staining exists and a type of the bacterium on an image that is based on the image data.

### CROSS-REFERENCE TO RELATED APPLICATIONS

This application is a Continuation of International Patent Application No. PCT/JP2021/025782, filed Jul. 8, 2021, which claims the benefit of Japanese Patent Applications No. 2020-123747, filed Jul. 20, 2020, and No. 2021-104154, filed Jun. 23, 2021, all of which are hereby incorporated by reference herein in their entirety.

### BACKGROUND OF THE INVENTION

### Field of the Invention

The present invention relates to a system for detecting bacteria in an image and classifying the bacteria.

### Background Art

In conventional infectious disease therapies, there has been a case where a wide variety of antibacterial agents are administered without identifying the bacterial species. If an antibacterial agent is unnecessarily administered, drug resistance bacteria resistant to the antibacterial agent are generated, which has become a social problem in recent years.

Examples of methods for solving such a problem include bacteria classification using Gram staining. By classifying bacteria by bacterial species based on Gram staining, it is possible to administer an antibacterial agent suitable for each bacterial species.

For example, Japanese Patent Application Laid-Open No. 2007-121282 discusses a technique for detecting bacteria in a specimen by using protein that links to the bacteria cell wall, and identifying whether the detected bacteria are Gram-positive or Gram-negative.

Japanese Patent Application Laid-Open No. 2007-232560 discusses a configuration of a Gram staining apparatus provided with a staining fluid and a cleaning fluid for quickly performing staining and cleaning operations in Gram staining.

Classifying bacteria by using Gram staining requires a series of operations including performing a staining operation, observing a specimen with a microscope to detect bacteria after the staining operation, and classifying the detected bacteria by shape and color.

Further, detecting the bacterial species from a Gram-stained specimen requires special knowledge and rich experience in Gram staining. This makes it necessary to rely on persons having such knowledge and experience, resulting in the concentration of burden on the specific persons.

### CITATION LIST

### Patent Literature

PTL 1: Japanese Patent Application Laid-Open No. 2007-121282
PTL 2: Japanese Patent Application Laid-Open No. 2007-232560

### SUMMARY OF THE INVENTION

According to an apsect of the present invention, an image processing apparatus includes an acquisition unit configured to acquire image data as result of imaging a Gram-stained specimen, and a generation unit configured to generate a display image by superimposing a position where a bacterium classified by Gram staining exists and a type of the bacterium on an image that is based on the image data.

Further features of the present invention will become apparent from the following description of exemplary embodiments with reference to the attached drawings.

### BRIEF DESCRIPTION OF THE DRAWINGS

FIG. 1 is a diagram illustrating a classification system for staining and classifying bacteria according to an exemplary embodiment of the present invention.

FIG. 2 is a block diagram illustrating configurations of a Gram staining apparatus, a working computer, and a server of the classification system according to an exemplary embodiment of the present invention.

FIG. 3A is a diagram illustrating a screen displayed on a display immediately after an application is activated.

FIG. 3B is a diagram illustrating a screen displayed on the display when a full automatic mode is selected.

FIG. 3C is a diagram illustrating a screen displayed on the display when the full automatic mode is started.

FIG. 4 is a flowchart illustrating processing in the full automatic mode of the Gram staining apparatus.

FIG. 5A is a diagram illustrating a glass slide on which a specimen is smeared.

FIG. 5B is a diagram illustrating a plurality of lattice-formed observation regions set on the glass slide.

FIG. 5C is a diagram illustrating an observation region.

FIG. 6 is a diagram illustrating a screen displaying results of bacteria detection and classification.

FIG. 7 is a flowchart illustrating processing for determining whether a region is suitable for bacteria classification.

FIG. 8 is a flowchart illustrating bacteria detection and classification processing.

FIG. 9A is a diagram illustrating a captured image.

FIG. 9B is a diagram illustrating data indicating information for each image.

FIG. 9C is a diagram illustrating data indicating positions, names of bacterial species, and reliability of detected bacteria.

FIG. 10 is a diagram illustrating a setting screen of an application executed on the working computer.

FIG. 11 is a flowchart illustrating processing to be performed when automatic transmission to the server is turned ON.

FIG. 12 is a diagram illustrating a screen for setting image transmission to the server.

FIG. 13 is a diagram illustrating a screen for setting a region to be subjected to bacteria detection and classification arbitrarily by a user.

FIG. 14 is a flowchart illustrating bacteria detection and classification processing to be performed on the region arbitrarily specified by the user.

FIG. 15 is a diagram illustrating a screen of an application for checking results of past detection and classification.

FIG. 16A is a diagram illustrating a screen displayed on the display when an individual mode is selected.

FIG. 16B is a diagram illustrating a screen displayed on the display when a specified operation is started.

FIG. 16C is a diagram illustrating a screen displayed on the display when an operation is completed.

### DESCRIPTION OF THE EMBODIMENTS

Exemplary embodiments of the present invention will be described in detail below with reference to the accompanying drawings.

A series of processing in which a doctor or a nurse collects a specimen from a patient, and classifies bacterial species through Gram staining will be described below.

In Gram staining, bacteria which are stained can be classified into four different types “GNR”, “GNC”, “GPR”, and “GPC” by color and shape. Further, “GPC” can be classified into two different types “GPC Chain” and “GPC Cluster” by shape. In this system, detected bacteria are classified into five different types: “GNR”, “GNC”, “GPR”, “GPC Chain”, and “GPC Cluster”. In bacteria detection and classification, general object detection by Deep Learning is used.

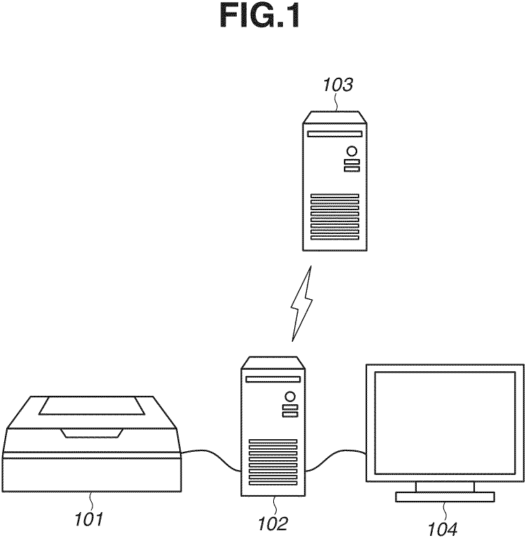

FIG. 1 is a diagram illustrating a classification system for staining and classifying bacteria according to an exemplary embodiment of the present invention. A Gram staining apparatus 101 serving as an image processing apparatus for staining and classifying bacteria is connected to an external working computer 102. The working computer 102 controls the Gram staining apparatus 101, and is wirelessly connected to a server 103 to access information on electronic medical charts of patients stored in the server 103. A display 104 connected to the working computer 102 displays bacteria classification results.

As preparation for performing Gram staining in the classification system illustrated in FIG. 1, a doctor or a nurse collects a specimen and then smear the specimen on a glass slide. This glass slide on which the specimen is smeared is set on the Gram staining apparatus 101. A methanol fluid, a Gram staining fluid, and a cleaning fluid are preset in the Gram staining apparatus 101.

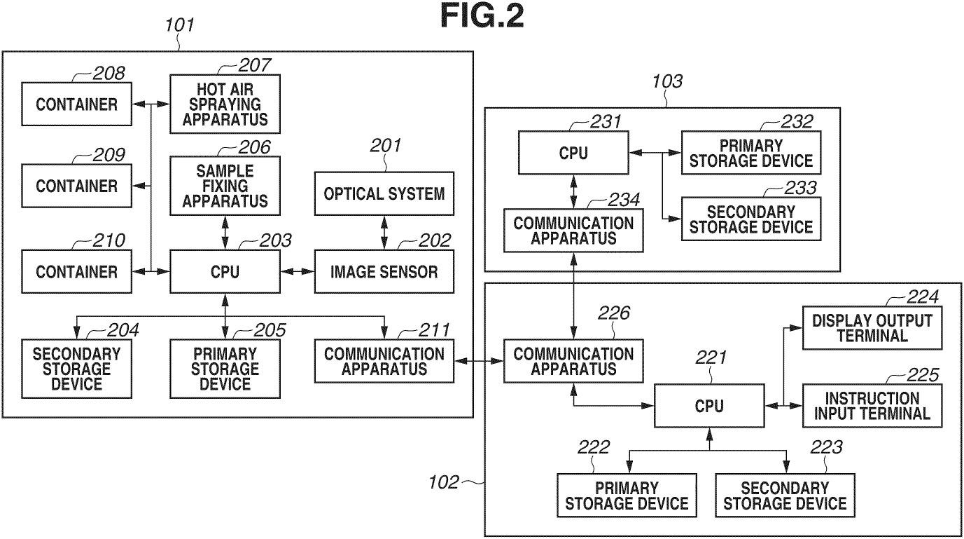

FIG. 2 is a block diagram illustrating configurations of the Gram staining apparatus 101, the working computer 102, and the server 103 of the classification system according to an exemplary embodiment of the present invention.

In the Gram staining apparatus 101, an optical system 201 includes a lens and a diaphragm and forms an image on an image sensor 202, such as a Charge Coupled Device (CCD) or Complementary Metal Oxide Semiconductor (CMOS) sensor, with a suitable amount of light from a subject. The image sensor 202 converts light that has passed through the optical system 201 and focused thereon into an image.

A central processing unit (CPU) 203 controls the operation of each component of the Gram staining apparatus 101. A secondary storage device 204 such as a hard disk drive stores programs used by the CPU 203 to control the operation of each component of the Gram staining apparatus 101. A primary storage device 205 such as a random access memory (RAM) stores a program loaded from the secondary storage device 204. The CPU 203 reads the program stored in the primary storage device 205.

The glass slide on which a specimen is smeared is fixedly placed on a sample fixing apparatus 206. The specimen is imaged by the optical system 201 and the image sensor 202. A hot air spraying apparatus 207 generates hot air to dry the specimen. A reservoir 208 is filled with the methanol fluid used to fix the specimen to the glass slide placed on the sample fixing apparatus 206. A reservoir 209 is filled with the Gram staining fluid used for Gram staining. A reservoir 210 is filled with the cleaning fluid used to clean the specimen during Gram staining. A communication apparatus 211 performs wireless or wired data communication with the working computer 102 for Gram staining.

The working computer 102 is configured by a personal computer or an edge computer and controls the operations of the Gram staining apparatus 101. The working computer 102 temporarily stores results of bacteria detection and classification performed by the Gram staining apparatus 101. A CPU 221 receives an instruction input by a user using a mouse, a keyboard, or a touch panel via an instruction input apparatus 225, and controls the operation of each component of the working computer 102. A secondary storage device 223 such as a hard disk drive stores a program used by the CPU 221 to control the operation of each component of the working computer 102. A primary storage device 222 such as a RAM stores a program loaded from the secondary storage device 223. The CPU 221 reads the program stored in the primary storage device 222.

The CPU 221 generates image and text data necessary for the user to operate an application for Gram staining, and transmits the generated image and text data to the display 104 via a display output terminal 224. Although the display 104 and the working computer 102 are described above as different apparatuses here, the working computer 102 may be provided with the display 104 like a tablet computer.

A communication apparatus 226 is wirelessly or wiredly connected to the Gram staining apparatus 101 and the server 103 and performs data communication therewith. The CPU 221 transmits an instruction related to the operations of the Gram staining apparatus 101 to the CPU 203 of the Gram staining apparatus 101 via the communication apparatus 226 and the communication apparatus 211.

The server 103 stores electronic medical charts. In a hospital, an electronic medical chart viewer application is installed in in-house computers used by doctors and nurses. The viewer application accesses the server 103 to acquire and display patient information. A CPU 231 controls the operation of each component of the server 103. A secondary storage device 233 such as a hard disk drive stores programs used by the CPU 231 to control the operation of each component of the server 103 and also stores electronic medical chart data as patient information. A primary storage device 232 such as a RAM stores a program and the electronic medical chart data loaded from the secondary storage device 233. The CPU 231 reads the program and data stored in the primary storage device 232. The CPU 231 receives a request from the CPU 221 of the working computer 102 via a communication apparatus 234 and transmits electronic medical chart data consistent with the request via the communication apparatus 234.

The working computer 102 is an image processing apparatus that activates an application for operating the Gram staining apparatus 101 in response to an instruction from the user.

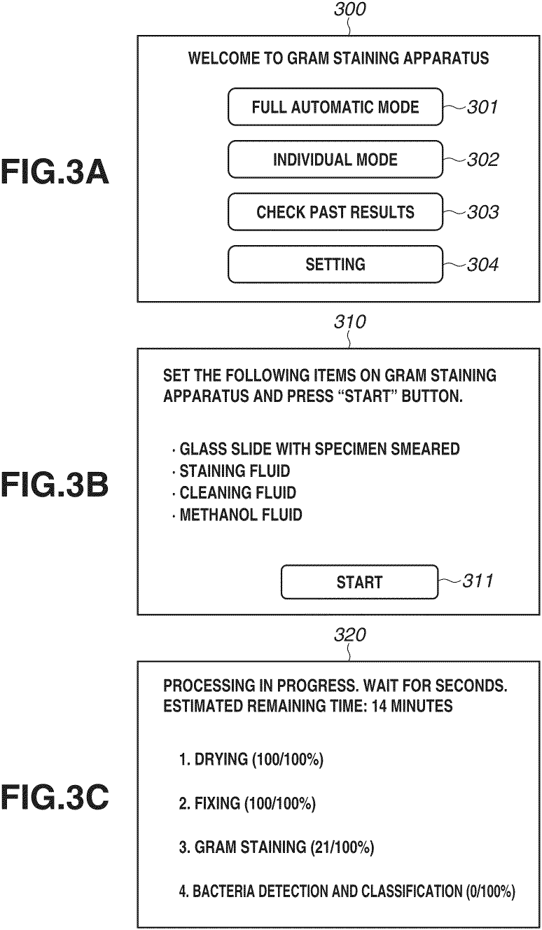

FIG. 3A is a diagram illustrating a screen 300 displayed on the display 104 immediately after the application is activated. FIG. 3B is a diagram illustrating a screen 310 displayed on the display 104 when a full automatic mode is selected. FIG. 3C is a diagram illustrating a screen 320 displayed on the display 104 when the full automatic mode is started.

The screen 300 displayed when the application is activated includes a “Full Automatic Mode” button 301, an “Individual Mode” button 302, a “Check Past Results” button 303, and a “Setting” button 304. The full automatic mode will be first described below.

When the user operates the instruction input apparatus 225 to select the button 301, the screen 310 illustrated in FIG. 3B appears on the display 104. The screen 310 displays materials that need to be set on the Gram staining apparatus 101. When all materials have been set and the user selects a “Start” button 311 on the screen 310, the Gram staining apparatus 101 starts the operation of Gram staining. When the “Start” button 311 is selected, the screen 320 illustrated in FIG. 3C appears on the display 104. The Gram staining apparatus 101 performs four different steps to complete bacteria detection and classification operations during which the screen 320 displays the progression rate of each step. The screen 320 also displays the remaining operation time until all steps are completed. Displaying the progress and the remaining time of the operation in this way enables enhancement of the usability of the user.

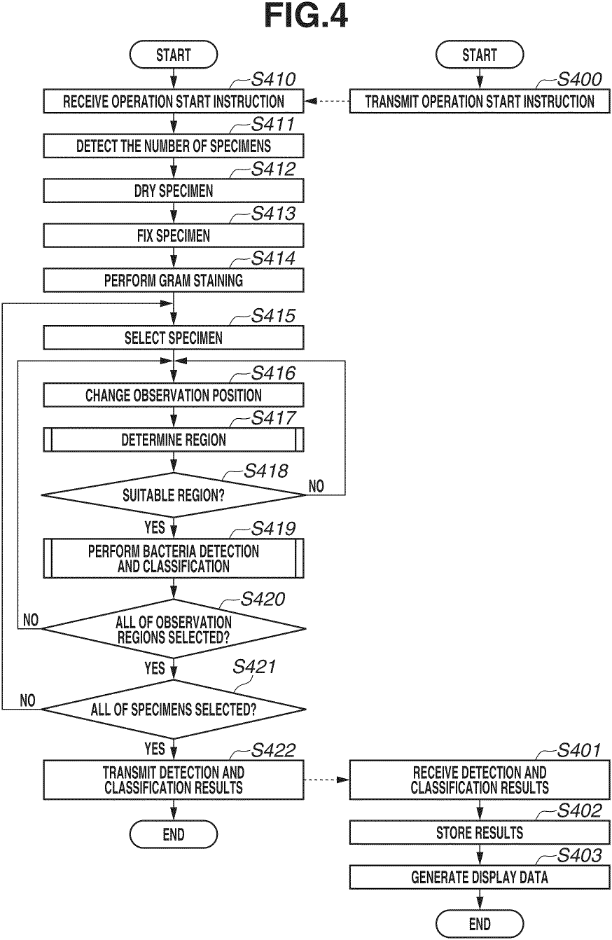

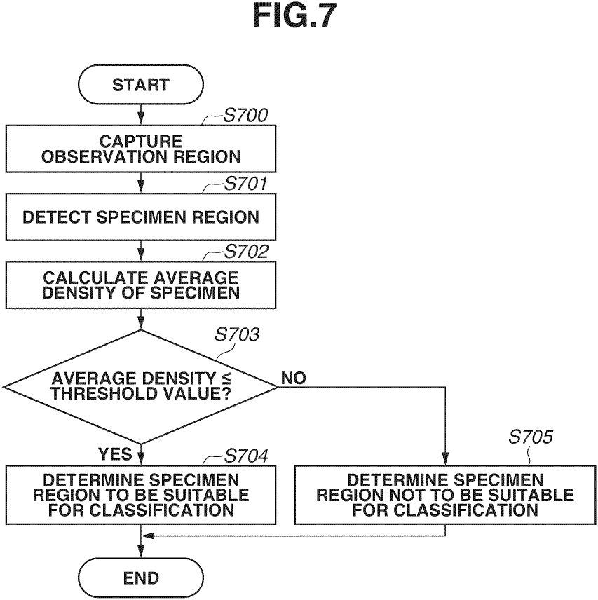

FIG. 4 is a flowchart illustrating processing performed when the Gram staining apparatus 101 operates in the full automatic mode. In FIG. 4, “step” is denoted as “S”. This also applies to FIGS. 7, 8, 11, and 14. In FIG. 4, steps S400 to S403 are processing performed by the working computer 102 under the control of the CPU 221, and steps S410 to S422 are processing performed by the Gram staining apparatus 101 under the control of the CPU 203.

In step S400, when the CPU 221 of the working computer 102 detects that the user selects the “Start” button 311, the CPU 221 transmits an operation start instruction to the CPU 203 of the Gram staining apparatus 101.

In step S410, the CPU 203 of the Gram staining apparatus 101 receives the operation start instruction from the CPU 221 of the working computer 102.

In step S411, the CPU 203 detects the number of glass slides set on the Gram staining apparatus 101. The CPU 203 may detect the number of glass slides by using an optical or mechanical sensor provided on the sample fixing apparatus 206 or by analyzing an image of the surface of the sample fixing apparatus 206 on which glass slides are placed. Alternatively, the user may input the number of glass slides.

In step S412, the hot air spraying apparatus 207 sprays hot air to the glass slides set on the sample fixing apparatus 206 to dry the specimens.

In step S413, the CPU 203 drops the methanol fluid contained in the reservoir 208 on the glass slides set on the sample fixing apparatus 206 by using an apparatus (not illustrated) and then fixes the specimens to the glass slides.

In step S414, the CPU 203 subjects the specimens on the glass slides to gram staining. In Gram staining, two different staining methods, Faber method and Bermy method, are often used. These two methods use different staining fluids but share common operation procedures. The methods use three to four different staining fluids. The specimens are stained with one staining fluid for a predetermined time and then cleaned. Then, the specimens are stained with another staining fluid and then cleaned again. The above-described procedure is repeated for the remaining staining fluids. In Gram staining, the Gram staining fluid contained in the reservoir 209 and the cleaning fluid contained in the reservoir 210 are used.

In steps S415 to S421, the specimens are sequentially observed. In step S415, the CPU 203 moves the sample fixing apparatus 206 to select the glass slide on which the first specimen is smeared. In the second and subsequent round of operations, the CPU 203 sequentially selects other glass slides in predetermined order.

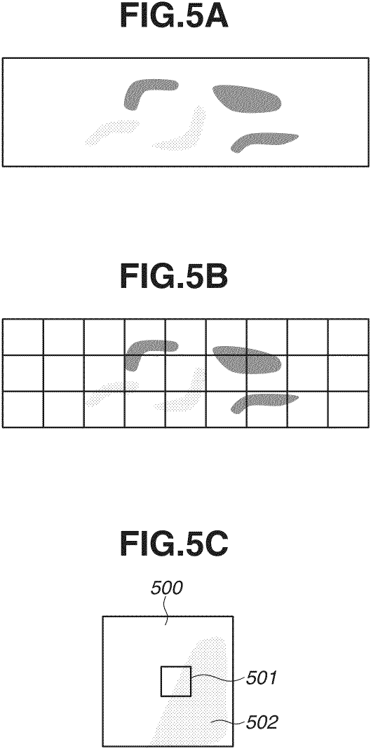

In step S416, the CPU 203 moves the sample fixing apparatus 206 to change the observation position with respect to the currently selected glass slide. FIG. 5A is a diagram illustrating the glass slide on which the specimen is smeared. FIG. 5B is a diagram illustrating a plurality of lattice-formed observation regions set on the glass slide. FIG. 5C is a diagram illustrating one observation region. The CPU 203 moves the sample fixing apparatus 206 so that the target to be observed by the optical system 201 and the image sensor 202 is sequentially aligned with each of the plurality of lattice-formed observation regions illustrated in FIG. 5B.

In step S417, the CPU 203 determines whether the selected observation region is suitable for bacteria classification. This region determination processing will be described in detail below.

In step S418, when the selected observation region is suitable for bacteria classification (YES in step S418), the processing proceeds to step S419. On the other hand, when the selected observation region is not suitable for bacteria classification (NO in step S418), the processing returns to step S416. In step S416, the CPU 203 selects the next observation region.

In step S419, the CPU 203 performs bacteria detection and classification. This bacteria detection and classification processing will be described in detail below.

In step S420, the CPU 203 determines whether all of the plurality of observation regions on the glass slide have been selected. When all of the observation regions have been selected (YES in step S420), the processing proceeds to step S421. On the other hand, when not all of the observation regions have been selected (NO in step S420), the processing returns to step S416.

In step S421, the CPU 203 determines whether all of the glass slides set on the sample fixing apparatus 206 have been selected. When all of the glass slides have been selected (YES in step S421), the processing proceeds to step S422. On the other hand, when not all of the glass slides have been selected (NO in step S421), the processing returns to step S415.

In step S422, the CPU 203 transmits bacteria detection and classification results to the working computer 102. Data to be transmitted will be described in detail below.

In step S401, the CPU 221 of the working computer 102 receives the bacteria detection and classification results via the communication apparatus 226.

In step S402, the CPU 221 stores the received results in the secondary storage device 223.

In step S403, the CPU 221 generates display data indicating the bacteria detection and classification results and displays the display data on the display 104 to allow the user to view the results.

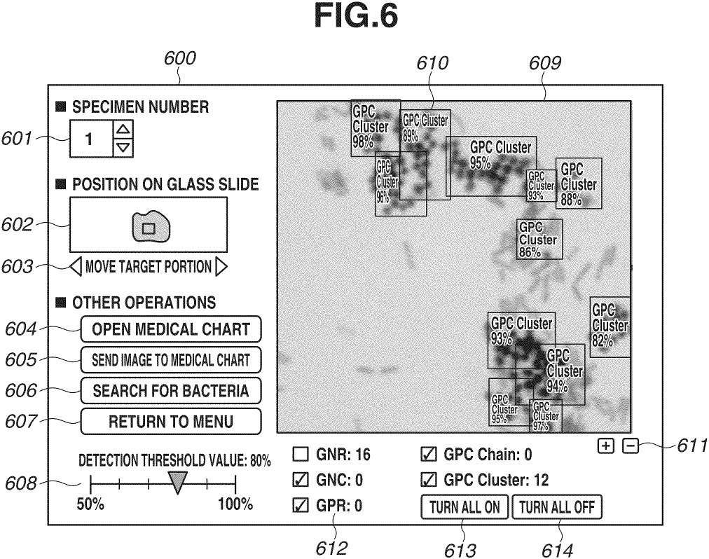

The results of bacteria detection and classification performed in the above-described manner are illustrated in FIG. 6. FIG. 6 is a diagram illustrating a screen for displaying the bacteria detection and classification results. A screen 600 displayed on the display 104 includes an image 609 of the imaged specimen. The image 609 includes a region 610 where bacteria are detected, and the region 610 is displayed with a frame indicating the region of the detected bacteria, a bacterial species, and reliability. The reliability indicates the reliability of a bacterial species classified by inference. The larger the value of the reliability, the higher the probability that the detected bacterial species is correct.

A bacterial species count 612 for each detected bacterial species is displayed below the image 609. The bacterial species count 612 indicates that the number of GNRs is 16, the number of GPC Clusters is 12, and the numbers of remaining bacteria are 0 in the image 609. There are check boxes next to the names of bacterial species, which enable the detection results to be filtered and displayed. In the screen 600, the detection result of GNR is not displayed since the check box of GNR is OFF. When the user presses a button 613 or 614, all of the check boxes are collectively turned ON or OFF, respectively.

The user can enlarge or reduce the displayed image 609 by operating buttons 611. A specimen number 601 is set for each specimen. The user can select a specimen by operating the up and down buttons displayed to the right of the specimen number 601. When three different glass slides are set on the Gram staining apparatus 101, any one of the three different specimens can be selected. When the specimen is switched to another, an image 602 indicating a position on the glass slide and the image 609 are updated. The image 602 indicates a position in the entire area of the glass slide which corresponds to the image 609. A button 603 is used to select another region on the same specimen that is determined to be suitable for bacteria classification. In FIG. 6, a region determined to be suitable for bacteria detection and classification is referred to as a “target portion”. Functions of buttons 604 to 607 will be described below. A slider bar 608 is used to change the threshold value of the reliability. Only the bacteria classification results with reliability equal to or higher than the threshold value set by the slider bar 608 are superimposed on the image 609.

FIG. 7 is a flowchart illustrating processing for determining whether the observation region is suitable for bacteria classification in step S417 in FIG. 4.

In the flowchart of FIG. 7, the CPU 203 determines whether the observation region is suitable for bacteria classification based on whether the specimen is thinly smeared or thickly smeared. To detect and classify the specimen, it is desirable that the specimen is thinly smeared. Thus, the CPU 203 calculates whether the specimen is thinly smeared and determines whether the observation region is suitable for bacteria classification.

In step S700, the CPU 203 drives the optical system 201 and the image sensor 202 to capture an image of the observation region.

In step S701, the CPU 203 detects a specimen region where bacteria exist in the observation region. In the image of an observation region 500 in FIG. 5C, a specimen region 501 is a region indicated in gray. The CPU 203 detects a specimen region by using pattern matching and the density difference from the glass slide.

In step S702, the CPU 203 calculates the average density of the specimen region 501. The CPU 203 obtains the average density to determine whether the specimen is thinly smeared.

In step S703, the CPU 203 determines whether the calculated average density is equal to or less than a threshold value. When the average density is equal to or less than the predetermined threshold (YES in step S703), then in step S704, the CPU 203 determines that the specimen region 501 is suitable for bacteria classification. On the other hand, when the average density is larger than the predetermined threshold (NO in step S703), then in step S705, the CPU 203 determines that the specimen region 501 is not suitable for bacteria classification.

When no specimen region is detected in the observation region in step S701, the CPU 203 determines that the observation region is not suitable for bacteria classification. The above-described method for determining a region suitable for bacteria classification is to be considered as illustrative, and other methods are also applicable. For example, the CPU 203 may determine whether the specimen region 501 is suitable for bacteria classification by using a trained model generated by machine learning in advance.

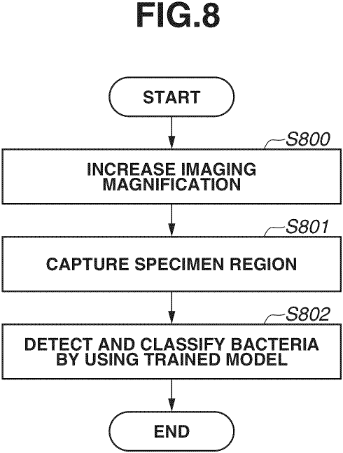

The flowchart in FIG. 8 will be next described below. FIG. 8 is a flowchart illustrating the processing for bacteria detection and classification in step S419 in FIG. 4. In the processing in FIG. 8, the imaging magnification is increased to the level at which bacteria can be actually imaged, the region determined to be suitable for bacteria classification is imaged at the imaging magnification, and then bacteria detection and classification are performed using machine learning-based inference.

In step 800, the CPU 203 increases the imaging magnification of the observation region. Generally, an imaging magnification of about 1,000 times is required to image bacteria, so that the CPU 203 drives the optical system 201 to increase the imaging magnification to 1,000 times.

In step 801, the CPU 203 images the specimen region 501 at the set imaging magnification by using the optical system 201 and the image sensor 202. For example, in a case of the observation region 500 in FIG. 5C, a region 502 in the observation region 500 is enlarged and imaged. For bacteria detection and classification, a boundary between specimen regions is suitable for observation. Therefore, the CPU 203 may perform processing for automatically detecting and imaging a region including a boundary between specimen regions by using pattern matching.

In step 802, the CPU 203 performs bacteria detection and classification. In this step, a technique for detecting target objects from a captured image and classifying the target objects by using a trained model obtained by performing machine learning based on Deep Learning. In general object detection based on Deep Learning, a learning image group labeled with a target object position is prepared in advance and machine learning is performed using the learning image group to create a trained model. Then, by causing the created trained model to read an image to be determined, target objects can be detected from the image and classified. In this system, a trained model that has been trained using a number of labeled images of Gram-stained bacteria is created, and the trained model is stored in the Gram staining apparatus 101.

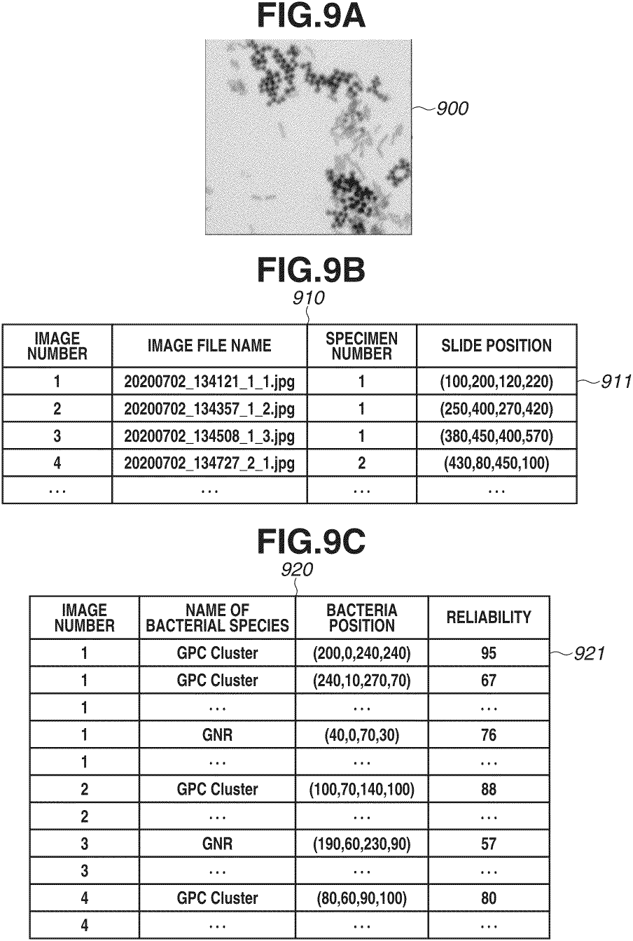

FIGS. 9A to 9C are diagrams illustrating data to be transmitted from the Gram staining apparatus 101 to the working computer 102 in the step S422 in FIG. 4. By transmitting data to the working computer 102 and storing the data in the working computer 102, the bacteria detection and classification results can be checked on the working computer 102 even after the power of the Gram staining apparatus 101 is turned off.

FIG. 9A illustrates an image 900 captured in step S801 in FIG. 8. This is the image of the region 502 in FIG. 5C. If there is a plurality of regions suitable for bacteria classification in one specimen, images are generated for the number of regions suitable for bacteria classification. The number of images increases with increasing number of specimens set on the Gram staining apparatus 101. Each image is given a file name.

FIG. 9B illustrates data 910 indicating information for each image. The amount of information included in the data 910 increases with increasing number of images. For example, information in a top row 911 indicates that the first image having a file name “20200702_134121_1_1.jpg”, and indicates the first specimen of which the imaging region exists at a position (100, 200, 120, 220) on the glass slide. This value indicates that the coordinates of the upper left and the lower right corners of the image are (100, 200) and (120, 220), respectively, on the glass slide. The image file name is used to associate with the captured image illustrated in FIG. 9A. The specimen number is used to display the specimen number 601 in FIG. 6. The position on the glass slide is used to generate the image 602 in FIG. 6.

FIG. 9C illustrates data 920 indicating the position of the detected bacteria, the name of a bacterial species, and the reliability. The amount of information increases with increasing number of detected bacteria. For example, information in a top row 921 indicates that GPC Cluster bacteria exist at a position (200, 0, 240, 240) in the first image and that the reliability is 95%. The name of a bacterial species, the position, and the reliability are used to display the bacteria detection and classification results on the image, like the regions 610.

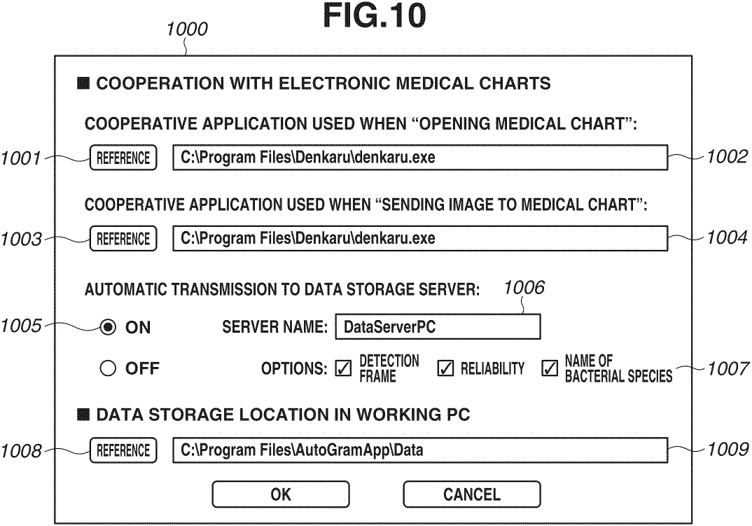

Referring back to FIG. 3, when the user selects the “Setting” button 304 in the screen 300, a screen 1000 illustrated in FIG. 10 appears. FIG. 10 is a diagram illustrating a setting screen of an application to be executed by the working computer 102.

The screen 1000 allows the user to make various settings of the application. This screen allows the user to set an application to be activated upon selection of an “Open Medical Chart” button 604 in FIG. 6. More specifically, the user selects a button 1001 displayed on the screen 1000 and then selects the file path of the electronic medical chart application. The selected file path is displayed in a field 1002. The screen also allows the user to set an application for transmitting an image upon selection of a “Send Image to Medical Chart” button 605 in FIG. 6. More specifically, the user selects a button 1003 displayed on the screen 1000 and then selects the file path of the electronic medical chart application. The selected file path is displayed in a field 1004.

A radio button 1005 having the options of ON and OFF is used to select whether to automatically transmit an image indicating the bacteria detection and classification results to the server 103 storing the electronic medical chart data, upon completion of bacteria classification in the full automatic mode. When the radio button 1005 is ON, data is automatically transmitted to the server 103 upon completion of the processing in the full automatic mode. When the radio button 1005 is OFF, data is not automatically transmitted.

When the option “ON” of the radio button 1005 is selected, a server name 1006 and image transmission options 1007 are enabled, and the image is transmitted in accordance with the server and options set here. A field 1006 is used to input a server name. The image transmission options 1007 allow the user to select whether to superimpose a “Detection Frame”, “Reliability”, and “Name of Bacterial Species” on the image when the image indicating the bacteria detection and classification results is transmitted. In order to change the data storage location in the working computer 102, the user uses a button 1008. The folder path of the data storage location is displayed in a field 1009.

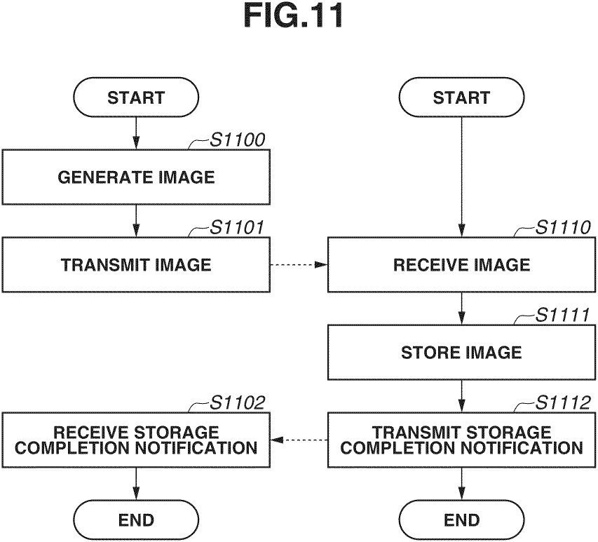

FIG. 11 is a flowchart illustrating processing to be performed when automatic transmission to the server 103 storing the electronic medical chart data is turned ON in the application setting screen illustrated in FIG. 10. More specifically, the processing illustrated in FIG. 11 is performed after completion of step S403 as the last processing in FIG. 4.

In FIG. 11, steps 1100 to 1102 are processing to be performed by the working computer 102 under the control of the CPU 221, and steps 1110 to 1112 are processing to be performed by the server 103 under the control of the CPU 231.

In step 1100, the CPU 221 of the working computer 102 generates an image in accordance with the settings of the transmission options 1007 illustrated in FIG. 10.

In step 1101, the working computer 102 transmits the generated image to the server 103 via the communication apparatus 226.

Next, in step 1110, the server 103 receives the image transmitted from the working computer 102, via the communication apparatus 234.

In step 1111, the CPU 231 of the server 103 stores the received image in the secondary storage device 233. When the received image is not provided with data for associating with the patient information in the electronic medical chart, the CPU 231 stores the image in the primary storage device 232 to allow a doctor and a nurse to associate the image stored in the primary storage device 232 with the patient information in the electronic medical chart afterwards.

In step 1112, the CPU 231 transmits a storage completion notification to the working computer 102.

In step 1102, the CPU 221 of the working computer 102 receives the storage completion notification. In this way, images can be automatically transmitted to the server 103 storing the electronic medical chart data.

Referring back to FIG. 6, buttons 604 to 607 will be described below. When the user selects the button 604, the electronic medical chart set in the field 1002 of the application setting screen in FIG. 10 is activated. When the user selects the button 607, the screen returns to the screen 300 illustrated in FIG. 3.

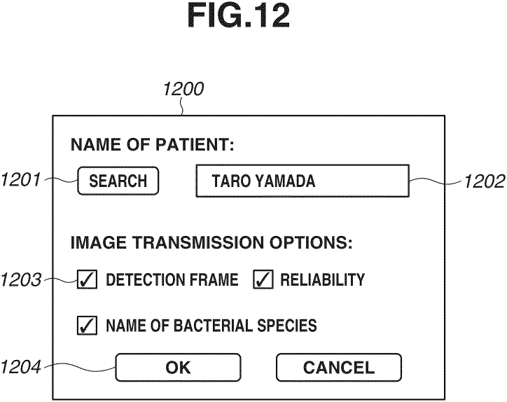

When the user selects the button 605, the screen transitions to a screen 1200 illustrated in FIG. 12. FIG. 12 is a diagram illustrating the 1200 screen for making settings for transmitting an image to the server 103. When the user operates a button 1201, names of patients in the electronic medical chart are displayed in list form on the screen, and the user selects a target patient from the list. The name of the selected patient is displayed in a field 1202. The user may input a part of the name of the patient in the field 1202 before starting the search with the button 1201.

Transmission options 1203 have functions similar to the functions of the transmission options 1007 in FIG. 10, and the image is transmitted in accordance with the settings of the options 1203. The transmission options 1203 are set for each user, and if the settings of the transmission options 1203 are different from the settings of the transmission options 1007, the settings of the transmission options 1203 are given priority. When the user operates an OK button 1204, the image is transmitted to the server 103 storing the electronic medical chart data in accordance with the set patient name and the set transmission options 1203, and the is incorporated in the electronic medical chart.

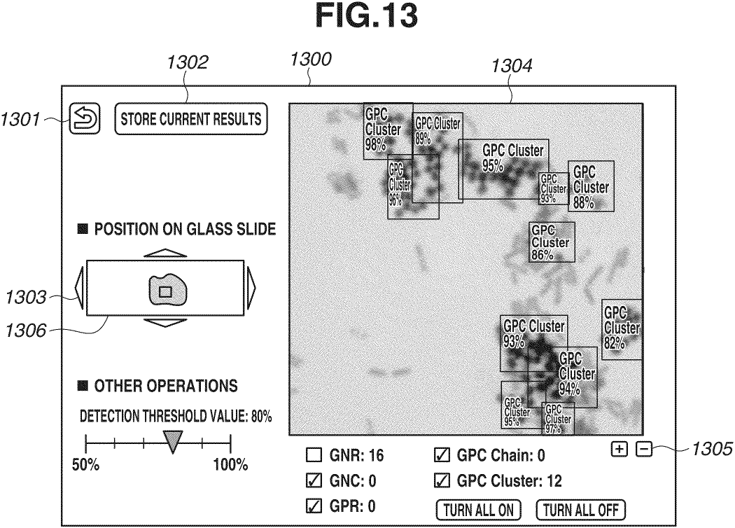

The button 606 in FIG. 6 will be described below. The button 606 is used by the user to set a desired region to be subjected to bacteria detection and classification. FIG. 13 is a diagram illustrating a screen for the user to set a desired region to be subjected to bacteria detection and classification. When the user operates the button 606, the screen transitions to a screen 1300 in FIG. 13. Since the screen 1300 has many portions common to the screen 600, only differences from the screen 600 will be described below. By using the screen 1300, the user specifies a desired observation region on the glass slide, and the Gram staining apparatus 101 performs bacteria detection and classification on the specified observation region. There are two different methods for changing the observation region to be subjected to bacteria detection and classification.

The first method is a method of moving the observation region to be displayed as an image 1304 by operating a mouse on the image 1304, like a typical image viewer. The user can increase or decrease the display magnification of the image 1304 by scrolling the mouse wheel. The image 1304 can also be enlarged or reduced by operating a button 1305. The second method is a method of moving the position of the observation region to be displayed on the glass slide by operating a button 1303. The user can also specify a desired position on an image 1306 of the entire glass slide to determine the observation region to be displayed as the image 1304.

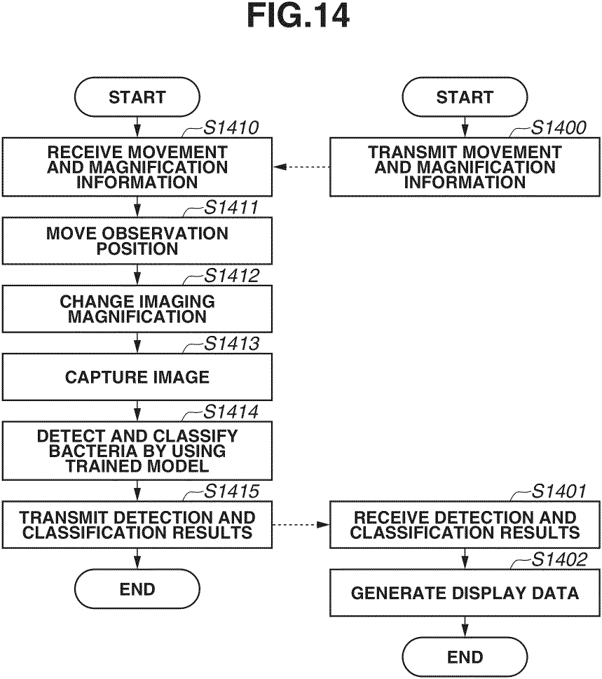

Each time the user changes the observation region on the glass slide to be displayed as the image 1304, the Gram staining apparatus 101 performs bacteria detection and classification. This processing will be described below with reference to FIG. 14. When the user operates a button 1301, the screen returns to the screen 600 in FIG. 6. When the user operates a button 1302, the currently displayed image and the bacteria detection and classification results are stored. In the full automatic mode, only results for regions automatically determined by the Gram staining apparatus 101 are stored. However, by operating the button 1302, the user can store results of bacteria detection and classification performed on a desired region.

FIG. 14 is a flowchart of processing for performing bacteria detection and classification on a desired region specified by the user. The working computer 102 performs processing in steps S1400 to S1402 under the control of the CPU 221, and the Gram staining apparatus 101 performs processing in steps S1410 to S1415 under the control of the CPU 203.

In step 1400, the CPU 221 of the working computer 102 transmits information about the movement of the observation region specified by the user and information about the magnification to the Gram staining apparatus 101.

In step 1410, the CPU 203 of the Gram staining apparatus 101 receives the information about the movement of the observation region and the information about the magnification.

In step 1411, in accordance with the information about the movement of the observation region, the CPU 203 drives the sample fixing apparatus 206 to move the imaging position on the glass slide.

In step 1412, the CPU 203 changes the imaging magnification of the optical system 201 in accordance with the information about the magnification.

In step 1413, the CPU 203 captures a still image via the image sensor 202.

In step 1414, the CPU 203 performs bacteria detection and classification by using a trained model based on a method similar to that in step 802 in FIG. 8.

In step 1415, the CPU 203 transmits the bacteria detection and classification results to the working computer 102 via the communication apparatus 211.

In step 1401, the CPU 221 of the working computer 102 receives the bacteria detection and classification results transmitted from the Gram staining apparatus 101, via the communication apparatus 226.

In step 1402, the CPU 221 generates display data indicating the bacteria detection and classification results and displays the display data on the display 104.

In the above-described manner, bacteria detection and a classification is also performed on a desired region specified by the user.

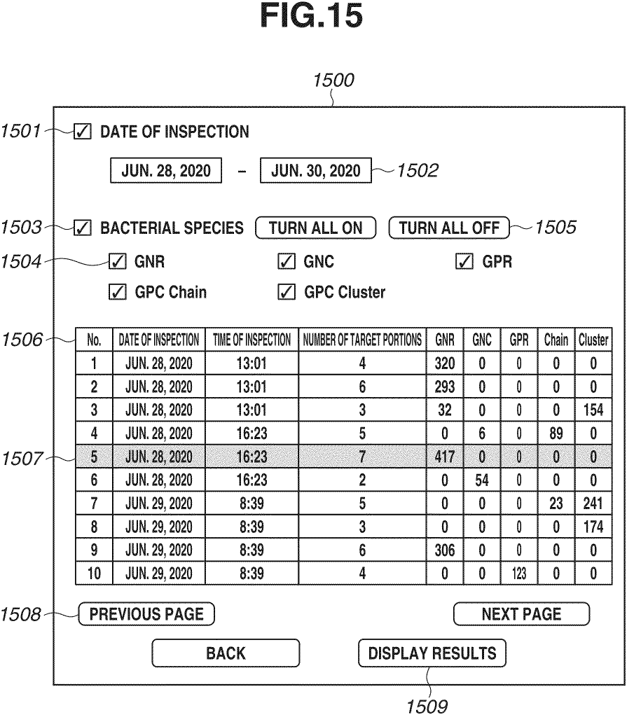

A method for checking past bacteria detection and classification results will be described below. The user can check the past bacteria detection and classification results by selecting the button 303 in the screen 300 in FIG. 3. FIG. 15 is a diagram illustrating a screen of an application for checking the past detection and classification results.

When the user selects the button 303 in FIG. 3, the screen transitions to a screen 1500 in FIG. 15. The screen 1500 allows the user to search and check the past bacteria detection and classification results. The search conditions include the date of inspection and the bacterial species. The date on which bacteria detection and classification are performed is handled as the date of inspection. To perform a search by the date of inspection, the user turns a check box 1501 ON and then specifies a range of dates of inspection in fields 1502. In the example of FIG. 15, the results of inspections performed on June, 28 to 30, 2020 are displayed.

A procedure for performing a search by a bacterial species will be described below. To perform a search by a bacterial species, the user turns a check box 1503 ON and then specifies bacterial species to be searched in the image by turning check boxes 1504 ON. In FIG. 15, since all of the check boxes 1504 of selectable bacterial species are turned ON, any image reflecting any of the bacterial species is to be subjected to a search.

Results of the search performed in the above-described manner are displayed in a list 1506. In the list 1506, search results are displayed for each specimen in a row. For example, for specimen No. 5 in a row 1507, an inspection for detecting and classifying is performed at 16:23 on Jun. 28, 2020, and the specimen has seven different target portions as regions suitable for bacteria detection and classification. The row 1507 also displays the number of bacteria for each bacterial species reflected in all of the regions suitable for bacteria detection and classification. It can be seen that, in specimen No. 5, 417 GNR bacteria are shown, and no other bacteria are shown.

In a case where there are many specimens, the user can switch the page of the list by using buttons 1508. When the user desires to display the details of a result, the user specifies a desired specimen from the list 1506 and then selects a button 1509, which causes the screen to transition to the screen 600 in FIG. 6.

Although, in the above-described full automatic mode, all of operations related to Gram staining are automatically performed, there may be cases in which the user wants to perform only a specific operation. In such a case, the user can specify a desired operation by selecting an individual mode.

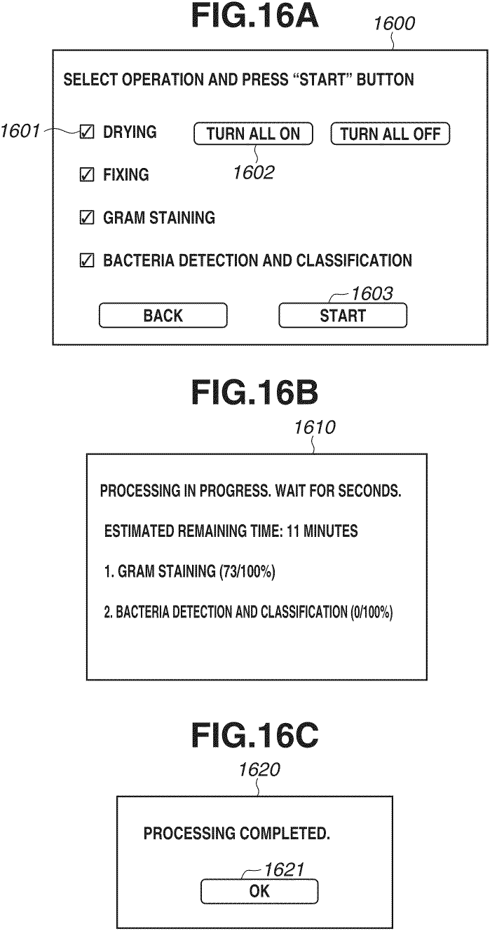

FIG. 16A is a diagram illustrating a screen 1600 displayed on the display 104 when the individual mode is selected. FIG. 16B is a diagram illustrating a screen 1610 displayed on the display 104 when the specified operation is started. FIG. 16C is a diagram illustrating a screen 1620 displayed on the display 104 when the operation is completed.

When the user selects the button 302 in the screen 300 in FIG. 3, the mode shifts to the individual mode, and the screen transitions to the screen 1600 in FIG. 16A. The screen 1600 allows the user to specify an operation to be performed, by turning a check box 1601 of the operation ON or OFF. The user can set all of the check boxes to ON or OFF by using buttons 1602. When the user selects an operation to be performed and then selects a Start button 1603, the selected operation is started. When the user selects the Start button 1603, the screen transitions to the screen 1610.

The screen 1610 allows the user to check the progression rate of processing and the remaining processing time, like the screen 320 in FIG. 3C. Upon completion of all operations, the screen transitions to the screen 1620 in FIG. 16C. When the user selects an OK button 1621 on the screen 1620, the screen transitions to the screen 300 in FIG. 3. In a case where the selected operations include “Bacteria Detection and Classification”, the screen transitions to the screen 600 in FIG. 6 upon completion of all of the selected operations, and the user can check the bacteria detection and classification results as in the full automatic mode.

While the present invention has specifically been described based on exemplary embodiments, the present invention is not limited to these specific exemplary embodiments, and various embodiments not departing from the spirit and scope of the present invention are also included in the present invention. Parts of the above-described exemplary embodiments may be suitably combined.

For example, an apparatus integrating the configurations of both the Gram staining apparatus 101 and the working computer 102 is also applicable. According to the above-described exemplary embodiments, the CPU 203 of the Gram staining apparatus 101 performs bacteria classification by using a learning model. However, the classification processing may be performed by the working computer 102 or the server 103.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

The present invention is not limited to the above-described exemplary embodiments but can be modified and changed in various ways without departing from the spirit and scope thereof. Therefore, the following claims are appended to disclose the scope of the present invention.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An image processing apparatus comprising:
an acquisition unit configured to acquire image data as a result of imaging a Gram-stained specimen; and
a generation unit configured to generate a display image by superimposing a position where a bacterium classified by Gram staining exists and a type of the bacterium on an image that is based on the image data.

2. The image processing apparatus according to claim 1, wherein the acquisition unit acquires information about the position where the bacterium classified by Gram staining exists and the type of the bacterium, together with the image data.

3. The image processing apparatus according to claim 1, wherein the generation unit further superimposes reliability of a classification result of the type of the bacterium on the image that is based on the image data.

4. The image processing apparatus according to claim 3, wherein the acquisition unit acquires information about the position where the bacterium classified by Gram staining exists and the type of the bacterium, and the reliability of the classification result of the type of the bacterium, together with the image data.

5. The image processing apparatus according to claim 4, further comprising a setting unit configured to set a threshold value of the reliability,
wherein, in a case where a bacterium has reliability equal to or higher than the threshold value, the generation unit superimposes the position where the bacterium exists and the type of the bacterium on the image that is based on the image data, and
wherein, in a case where a bacterium has reliability lower than the threshold value, the generation unit does not superimpose the position where the bacterium exists and the type of the bacterium on the image that is based on the image data.

6. The image processing apparatus according to claim 5, wherein the setting unit changes the threshold value based on a user instruction.

7. The image processing apparatus according to claim 1, further comprising a setting unit configured to set, for each type of a bacterium, whether to superimpose the position where the bacterium exists and the type of the bacterium on the image that is based on the image data.

8. The image processing apparatus according to claim 7, wherein the setting unit sets a type of a bacterium for which the position where the bacterium exists and the type of the bacterium are to be superimposed, based on a user instruction.

9. The image processing apparatus according to claim 1, further comprising a communication unit configured to transmit data on the display image generated by the generation unit to a server having electronic medical charts.

10. The image processing apparatus according to claim 9, further comprising a determination unit configured to determine a region to be imaged of the Gram-stained specimen, based on a user instruction,
wherein the communication unit transmits information indicating the region to be imaged to an imaging apparatus that images the Gram-stained specimen.

11. An image processing method comprising:
acquiring image data as a result of imaging a Gram-stained specimen; and
generating a display image by superimposing a position where a bacterium classified by Gram staining exists and a type of the bacterium on an image that is based on the image data.

12. A non-transitory storage medium storing a program for causing a computer to execute the image processing method according to claim 11.

13. The image processing apparatus according to claim 1, wherein information indicating the position where the bacterium classified by Gram staining exists and the type of the bacterium is information acquired by using a trained model generated by machine learning using the image data.
